(19) United States
(12) Patent Application Publication
(10) Pub. No.: US 2003/0013634 A1
(43) Pub. Date: Jan. 16, 2003

(54) Synthesis of small particles

(76) Inventors: Neil Russell Foster, Cherrybrook (AU); Hubert Leonardus Regtop, Mittagong (AU); Fariba Dehghani, Rosebery (AU); Rana Taysir Bastami, Greenacre (AU); Hak-Kim Chan, North Sydney (AU)

Correspondence Address: GARY CARY WARE & FRIENDENRICH LLP, 4365 EXECUTIVE DRIVE, SUITE 1600, SAN DIEGO, CA, 92121-2189, US

(21) Appl. No.: 10/017,135

(22) Filed: Dec. 7, 2001

(30) Foreign Application Priority Data
Dec. 8, 2000 (AU) PR1970

(51) Int. Cl. A61K 31/00; B29B 9/00

(52) U.S. Cl. 514/1; 264/5

(57) ABSTRACT

The invention provides a method for forming fine particles of a substance, the method including contacting a non-gaseous fluid containing the substance with a dense gas to expand the fluid, the dense gas including (a) an anti-solvent and (b) a modifying agent which modifies the polarity of the anti-solvent, and particles formed by the method.

This application claims priority from Australian provisional patent application no. PR1970 filed on Dec. 8, 2000, herein incorporated by reference in it's entirety.

### FIELD

The present invention relates to a method for forming fine particles of a substance, in particular, pH-sensitive proteins such as insulin, by anti-solvent precipitation. The invention also relates to fine particles of a pH-sensitive, biologically active substances produced by the method and to compositions, particularly pharmaceutical compositions, containing a pH-sensitive, biologically active substance.

### BACKGROUND

Production of uniform micron size particles (or within a narrow size range) of fragile molecules such as proteins is a challenge in the pharmaceutical industry.

One use of fine particles is pulmonary absorption of drugs. This is an important route of entry for many indications including some pulmonary diseases, for example, bronchial asthma. One advantage of this mode of administration is that access to the circulation is rapid, because the surface area is large. As well as almost instantaneous absorption of the drug into the blood, delivery to the lung has the advantages of avoidance of hepatic first-pass loss, and in the case of pulmonary disease, local application at the desired site of action.

Delivery to the lung may also provide an alternative for the treatment of conditions that have traditionally been treated by systemic administration of a drug. The administration of proteins is a case in point. Insulin is currently administered by injection because it is not stable in the gastrointestinal tract. Diabetic patients need to self-administer several injections. However, there is a lack of compliance with the use of injections because of the associated inconvenience and pain. Administration of the protein to the lung is more likely to be accepted by such patients and is therefore an attractive alternative to injections, as long as the protein can be formed as fine particles, without significant loss of biological activity. Usual criteria for the use of aerosol delivery for the administration of therapeutic drugs to the lung are that the drug is in particulate form with the particles having a size in the range of about 0.05-10 μm, preferably 1-5 μm while (obviously) retaining biological activity, which often requires the substance's structure to be maintained. A common problem in manufacture of such particles is unacceptable variation in particle size.

Drugs in the form of fine particles are also suitable for use in the area of oral, controlled or sustained release delivery. One application of such technology is in the case of a drug in which there is a small difference in dosage levels between the drug being effective and being toxic. In the latter technology, it is also important that the particles have a uniform particle size.

Another application of fine particles of pharmaceuticals is transdermal drug delivery. Apart from traditional sub-cutaneous, intravenous, etc. injection, new methods of administration are being used, such as lasers to create a fine channel through the skin for drug delivery. A similar mechanism involving high-pressure drug delivery transdermally is also being used. Thus, the applications for fine or micron-sized pharmaceutical particles are increasing.

Dense gas techniques utilizing fluids, near or above their critical point, as a solvent or anti-solvent have been developed in recent years. Two dense gas methods have been considered for the production of solid particles. The first method is known as the Rapid Expansion of Supercritical Solutions (RESS), and involves expanding a supercritical solution of the material of interest through a nozzle. Whilst providing an effective method for producing some fine particles, the applicability of the RESS method is limited by the low solubility of proteins in dense carbon dioxide.

The second method, known as the gas anti-solvent process, involves rapidly precipitating solutes from organic solutions, typically using dense carbon dioxide as an anti-solvent. The anti-solvent expands the solution, thereby decreasing the solvation power of the solvent, and eventually resulting in the precipitation of the solute.

Gas anti-solvent processes have been utilized for the generation of micron-sized particles in two modes. The first mode, known simply as the gas anti-solvent process (GAS), involves the gradual addition of an anti-solvent to the organic solution containing the solute until the precipitation occurs. The second mode, known as the Aerosol Solvent Extraction System (ASES), involves continuous introduction of a solution containing the solute of interest through a nozzle into a flowing dense gas stream. As the solution is sprayed in to the dense gas, high degrees of supersaturation result in the precipitation of fine particles. In general, precipitation using this process is rapid and requires mild operating temperatures and pressures.

The GAS process has been attempted for the generation of micron-sized particles of insulin, lysozyme, and peroxidase. The difficulty of applying these techniques to the production of micronised particles of pH sensitive proteins is that they involve exposure of the protein to organic solvents, the latter being potential denaturants. This would, for example, inactivate insulin. Organic solvents are also undesirable as they are more difficult to dispose of Thus, this process is largely unsuitable.

In one attempt to overcome this limitation, a form of the ASES process has been developed, referred to as Solution Enhanced Dispersion by Supercritical Fluid (SEDS). SEDS involves using the ASES process but with a special coaxial nozzle which, in part, overcomes the problem of exposure to organic solvents. This technique facilitates the generation of bioactive particles of lysozyme from aqueous solutions, the advantage of which is that water is the native and favourable medium for proteins. However, denaturation of pH sensitive substances can occur given the aqueous carrier, by changes to the pH during the particle generation.

We have found that the biological activity of some proteins may be adversely affected when CO₂ is used as the anti-solvent. We believe that the dissolution of CO₂ in the aqueous solution decreases the pH of the aqueous phase to an extent that may influence the biological activity and/or structure of pH-sensitive proteins such as insulin.

AstraZeneca AB WO 00/30612 discloses a method of producing particles of a drug which is susceptible to pH degradation. Using a SEDS process, an acid-labile substance in a hydrated form is said to be obtained without substantial degradation of the product. The process involves dissolution of the substance in a solvent or mixture of solvents (an organic solvent, for example an alcohol, ether or ketone). This solution is then contacted with a dense gas to extract the organic solvent and precipitate the fine particles. The solvents can also be added to the process as modifiers or cosolvents. However, this method also involves the substance of interest being in solution with an alcohol, ether or ketone, which will usually denature a pH sensitive molecule.

Current apparatus for the production and collection of particulate products comprise a precipitator and a collection device in the same vessel. The solution containing the product of interest and the anti-solvent (which contains the dense gas and, optionally, a modifier) are passed through the precipitation vessel co-currently. As the particles are formed, they fall to the bottom of the collection device under gravity and can become compacted, aggregated (physical association) or agglomerated (chemically bonded). The particles can also become further compacted during the washing stage at the end of the process, due, for example, to the high pressure and high flow-rate of the dense gas anti-solvent.

Aggregation occurs when a collection of two or more particles are held together by weak cohesive forces, such as van der Waal's forces. Aggregates can be dispersed with shear forces and/or solvents. Agglomeration on the other hand, occurs when a collection of two or more particles are held together by strong inter-particle forces such as crystal bonds. Agglomerates are more difficult to break up and disperse.

In small particle formation processes, it is desirable to avoid the particles becoming agglomerated or compacted, since it is more difficult to break this material up, particularly while avoiding damage to the active component. The particles resulting from such processes are, therefore, not uniform in size and shape, which is not ideal for the use of such particles in pharmaceutical applications. However, some degree of aggregation may be desirable in some situations where the particles produced are too fine to be collected. The fine powders that have not become aggregated may be washed out of the system, resulting in a low yield. Aggregation between particles makes the particles larger and easier to collect, and after collection the aggregate can be broken up by mechanical force.

Particles to be used for the pulmonary delivery of pharmaceuticals should ideally be less than 5 to 10 μm in diameter. Particles of this size are easy to aerosolise, and when inhaled, these particles are easily able to reach the lungs. However, when particles become compacted in the collection vessel, the mass fraction of particles with a diameter of less than 5 μm (and thus suitable for pulmonary delivery) is low.

Collection processes using known single-stage apparatus are essentially batch processes with short run times, due to the necessity of regularly stopping the run to remove the precipitated particles before caking occurs. The production of particles using such apparatus is thus, necessarily, a batch-wise process. The process is therefore inefficient and there can be poor yields and recovery of the product.

Investigations have been carried out to overcome the problems associated with generating fine, relatively uniform particles of a pH sensitive substance while maintaining its structure and/or activity, particularly biological activity.

The investigations were also carried out to design an apparatus for particle formation which operates in a more efficient manner and does not damage the particles that are formed using the apparatus or substantially increase the average particle size.

### SUMMARY OF THE INVENTION

In a first aspect, the present invention provides a method of forming fine particles of a substance, the method comprising: contacting a non-gaseous fluid containing the substance with a dense gas to expand the fluid, the dense gas including (a) an anti-solvent and (b) a modifying agent which modifies the polarity of the anti-solvent.

Preferably, the anti-solvent does not significantly alter the pH of the non-gaseous fluid.

The method of the present invention is capable of producing fine particles of the substance, and is particularly useful for the production of fine particles of pH sensitive substances and biologically active substances, since the biological activity of such substances is substantially retained. The present method is also particularly suited to water soluble substances.

The non-gaseous fluid is preferably an aqueous solution.

The anti-solvent used in the method of the invention should be a neutral solvent and/or a solvent of relatively low polarity. Suitable solvents include a C₁₋₄ alkane gas, a C₂₋₄ alkene gas, a C₂₋₄ alkyne gas, hydrofluorocarbons, refrigerants, like RF134a, and some organic solvents, such as hexane, or two or more thereof. Preferably the anti-solvent is an alkane gas. Ethane is a particularly preferred anti-solvent.

The modifying agent may be present in an amount sufficient to absorb or extract substantially all of the non-gaseous fluid of the non-gaseous fluid-biologically active substance solution.

The modifying agent may be any substance that modifies the polarity of the anti-solvent and acts as an extractant for (ie. solvent for) the non-gaseous fluid. The modifying agent may be selected from the group consisting of C₁₋₆ alkanols, C₁₋₆ thiols and C₁₋₆ amines. Preferably, the modifying agent is ethanol.

A particularly preferred anti-solvent/modifying agent combination is ethane/ethanol.

The dense gas can be at various temperatures and pressures. Preferably the temperature of the dense gas is in the range of −20° C. to about 100° C., most preferably about 5° C. to about 45° C. The lower temperatures result in increased viscosity an reduced mass transfer properties, and this reduces efficiency of the method. High temperatures are more costly to run and may damage the substance. Preferably the dense gas has a pressure in the range of about 1 bar to 400 bar. A pressure between about 5 to 200 bar is particularly preferred. Most preferably, the pressure of the dense gas is such as to maintain the mixture of solvent, anti-solvent and modifying agent as a single phase which reduces loss of precipitate which may remain dissolved in a second phase, and be washed from the system.

Preferably, both the anti-solvent gas and the modifier are substantially inert to the pH-sensitive, biologically active substance.

The particles produced by the method of the invention may also include delivery agents such as liposomes, lipids (including phospholipids), water soluble polymers, controlled-delivery coatings, surfactants, phytosterols, and any other delivery agents known in the art.

Preferably, substantially all of the fine particles produced by the method of the invention have a particle size less than 10,000 nm. More preferably, the fine particles have a size no greater than 6,500 nm. Particles having a size in the range of up to 5,000 nm are particularly useful for administration to the lung. If smaller particles are desired, it is believed that the method of the present invention can produce particles down to nanometer size, although such particles can be difficult to collect and naturally aggregate into larger particles.

Preferably, about 50% of the particles formed are between 625 and 10,000 nanometers across.

The solution of the pH-sensitive, biologically active substance may be contacted with dense gas in any suitable manner. Preferably, the solution is introduced as droplets into the dense gas. For example, the solution and dense gas may be contacted by concurrently spraying the two through a nozzle or the like. Alternatively, the solution may be sprayed through the dense gas. A further option is to pass the solution concurrently or countercurrently with respect to a stream of the dense gas. The solution may be passed through a continuum of the dense gas in the form of a thin film or plurality of streams.

Preferably the method of the invention is carried out using the ASES process. The term “pH-sensitive, biologically active substance”, as used throughout the specification, refers to any natural or synthetic substance which possesses a biological activity such as, for example, an enzymatic activity, channel function (e.g. ion channel), receptor or binding function, hormonal or neurotransmitter activity, or other pharmacological activity, or a protein, polypeptide, peptide, peptide analog or peptidomimetic, or nucleic acid or nucleic acid in association with a protein, polypeptide or peptide, which is adversely affected by pH outside of the normal physiological pH range (e.g. 6.8 to 7.5), especially low pH (e.g. less than 5.0). The adverse affect upon the biological activity caused by the pH may be the result of, for example, degradation, cleavage or conformational changes in the substance or inactivation of an active site or binding domain.

The pH-sensitive, biologically active substance is preferably selected from the group consisting of an antimicrobial agent, virus, antiviral agent, antifungal pharmaceutical, antibiotic, nucleotide, DNA, antisense DNA, RNA, antisense RNA, amino acid, peptide, protein, enzyme, hormones, immune suppressant, protease inhibitors, thrombolytic anticoagulant, central nervous system stimulant, decongestant, diuretic vasodilator, antipsychotic, neurotransmitter, sedative, anaesthetic, surfactant, analgesic, anticancer agent, anti-inflammatory, antioxidant, antihistamine, vitamin, mineral, sterol, phytosterol, lipid and esters of fatty acids.

More preferably, the pH-sensitive, biologically active substance is selected from proteins, polypeptides, peptides, peptide analogs or peptide mimetics. Most preferably, the pH-sensitive, biologically active substance is selected from the proteins insulin, erythropoetin, calcitonin, LHRH, somatostain, epidermal growth factors, DNase platelet derived growth factors, interleukins, interferons, cytokines, peptides of immunoglobulins, TNF and other biologically active peptides, monoclonal antibodies based on TNF inhibitors as well as antibodies based on inhibitors of cytokines and interleukins.

In a second aspect, the present invention provides a pharmaceutical composition comprising particles of a pH-sensitive, biologically active substance produced by the method of the present invention.

The pharmaceutical composition is preferably in a form suitable for inhalation delivery, for example, for delivery by a metered dose inhaler or a nebuliser. Further, a transdermal delivery system may be used (eg, recent devices involving laser-generated or high-pressure dermal channels) and more traditional parenteral administration.

In a third aspect, the present invention provides a method of treatment of a subject, the method comprising administering to the subject, an effective amount of particles of a pH-sensitive, biologically active substance produced by the method of the present invention.

Preferably the administration of particles of a biologically active substance is by inhalation, preferably as an aerosol.

The method of the third aspect may be the treatment of insulin-dependent diabetes by administration of insulin particles produced by the method of the present invention.

In a fourth aspect, the invention provides smaller particle than are possible in the prior art by use of a neutral antisolvent modified by a modifier to change its polarity by using a separate collection vessel in a “dual stage” process, particularly where the newly formed fine particles are largely suspended within the vessel by the force of the dense fluid flowing through the particles being balanced by gravity in the opposite direction, to reduce aggregation and agglomeration of the particles.

In the description, the term “dense gas” means a fluid substantially near or above its critical pressure (Pc) and temperature (Tc). In practice, the pressure of the fluid is likely to be in the range (0.5-1.5)Pc and its temperature (0.5-1.2)Tc.

It will be understood that the term “comprises” (or its grammatical variants) as used in this specification is equivalent to the term “includes” and should not be taken as excluding the presence of other elements or features.

The method of the present invention, in its preferred forms, may provide one or more of the following advantages:

1. The ability to produce fine powders of proteins and other pharmaceuticals with narrow particle size distributions.

2. The ability to use aqueous solutions thereby enabling concentrated solutions of material to be processed with minimal risk of deactivation of biological activity. Aqueous solutions are also easier and cheaper to handle.

3. The use of one of the preferred anti-solvents, ethane, overcomes the problems associated with an acidic pH environment (ethane is neutral).

4. The use of an organic compound such as ethanol as the modifier in the ethane phase appears to enhance the morphological characteristics of the powders produced, including insulin. While not wishing to be bound by any particular theory, it appears that the morphological characteristics of the powders produced are also dependent on the relative concentrations of the solutions at the time of contact, the time period that the solutions are in contact with each other, and the time period that the particles are in contact with each other after precipitation. These variables can be adjusted during use of the method to optimise results.

5. The ability to use a substance such as ethanol as a modifier for the anti-solvent as described in 3 above, yet produce fine particles of a biologically active substance in which 98 to 100% biological activity has been retained.

6. The ability to introduce an additional component in either the solvent stream or the modified anti-solvent stream, which when co-precipitated with the protein or pharmaceutical will enhance dissolution rates and/or bioavailability.

7. The ability to process materials at temperatures below those required for supercriticality, thereby reducing the risk of thermal degradation.

8. The ability to work at lower pressures than that claimed in the prior art, thereby reducing the potential cost of the process.

Without being bound by any particular theory, it is suspected that the effect of CO₂ on the pH of the aqueous solution is eliminated because ethane modified with ethanol is used as the anti-solvent. It was found that use of this anti-solvent/modifier combination did not significantly degrade the biological activity of the insulin precipitated from aqueous solution.

Another aspect of the invention provides an apparatus wherein the precipitator and the collector are two separate vessels, in which fine particles (with a narrow particle size distribution) can be produced which are not affected by the problems of compaction, agglomeration and aggregation. In addition, the particles are finer than particles produced using prior art apparatus, and the mass fraction of particles with a diameter of <5 μm is higher than in prior art apparatus. Also, an essentially batch process can be made semi-continuous.

Within the collector device, the particles are suspended by the force exerted on them by a flow of dense gas, which force is generally equal and opposite to the particles' weight. Therefore, the newly formed particles do not fall on top of the previously formed particles and are not subjected to a weight which could deform their shape and are then less susceptible to aggregation.

Further, the use of such an apparatus allows higher yield and recovery of particles per run, the ability to process more material per run with longer run times, all of which lead to a more efficient process and greater production capacity.

Such an apparatus can be readily scaled up to process larger amounts of material.

In order that the invention may be more readily understood, we provide the following non-limiting embodiments as examples.

### BRIEF DESCRIPTION OF DRAWINGS

FIG. 1 is a schematic diagram of an embodiment of an apparatus that may be used in the process of the present invention.

FIG. 2 shows scanning electron micrograph (SEM) images of insulin.

FIG. 2(a) is an image of freeze-dried insulin, where the particles have a plate-like morphology.

FIG. 2(b) shows the spherical insulin particles produced using the method of the present invention at 45° C. and 155 bar.

FIG. 3 shows SEM images of insulin particles produced at 25° C. and 155 bar, using as the anti-solvent (a) carbon dioxide modified with ethanol; and (b) ethane modified with ethanol.

FIG. 4 shows a graph comparing the aerodynamic particle size distribution of insulin particles precipitated at 25° C. and 150 bar using 20 mol % ethanol in carbon dioxide and 30 mol % ethanol in ethane.

FIG. 5 shows an HPLC chromatogram of the insulin monomer.

FIG. 6 shows an HPLC chromatogram for the separation of insulin and deamido insulin.

FIG. 7 shows an SEM of insulin particles.

FIG. 8 shows a graph which represents the aerodynamic particle size distribution of insulin powder.

FIG. 9a shows a schematic diagram of a common production scale ASES apparatus, with a precipitation vessel which has a filter at the bottom of the vessel to prevent the particles being carried out of the vessel.

FIG. 9b shows a laboratory scale version of the apparatus represented in FIG. 1a.

FIGS. 10a (production scale) and 10b (laboratory scale) illustrate another embodiment of the invention, having a separate particle collection vessel, also called a “dual stage” apparatus.

FIG. 11 describes another embodiment of the invention, an apparatus having more than one particle collection vessel.

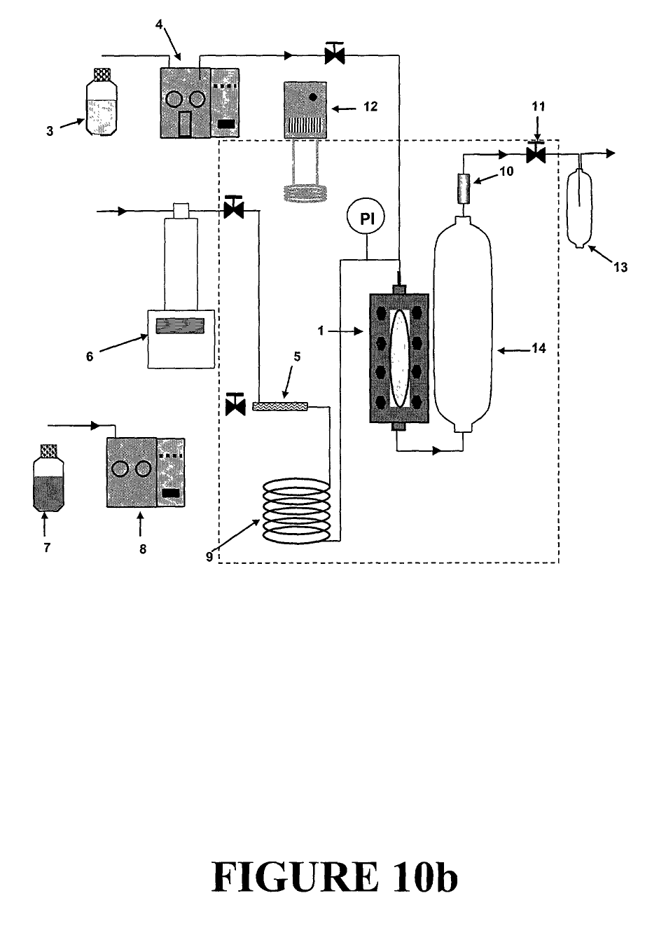

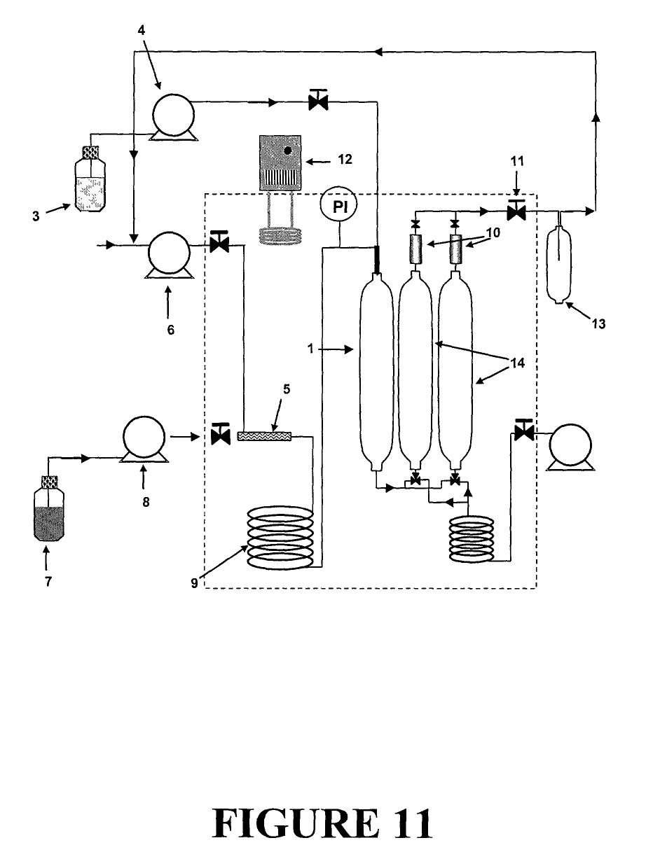

The set-up for the ASES apparatus (FIGS. 10a, 10b and 11) was designed to improve the proportion of fine particles generated of the drug, which could be collected as such, and increase the yield and recovery of the product. In order to minimize the compaction and increase the fine particle mass (FPM) fraction of the precipitate, a dual precipitation and collection vessel arrangement with no filter between the precipitation and collection vessels was used. The precipitates and anti-solvent pass co-currently from the first vessel and counter-currently in the second vessel. The embodiment in FIG. 11 has “parallel” collection vessels to enable continuous (rather than batch) operation by alternating between the two collection vessels.

While suitable for any of the proteins mentioned above, the examples illustrating the invention are described using insulin as the desired active ingredient. Similarly, for the purposes of illustration, the examples describe the use of ethane as the anti-solvent and ethanol as the modifier.

### EXAMPLES

Materials

Low endotoxin bovine insulin (lyophilized powder of 28.5 USP units/mg, lot No. 47H0573) and sodium hydroxide were purchased from Sigma Chemicals and used as received and dissolved in deionised water. Liquid carbon dioxide and ethane (Industrial Grade 99.95%) were purchased from BOC Gases.

### Example 1

### Standard ASES Apparatus

Procedure

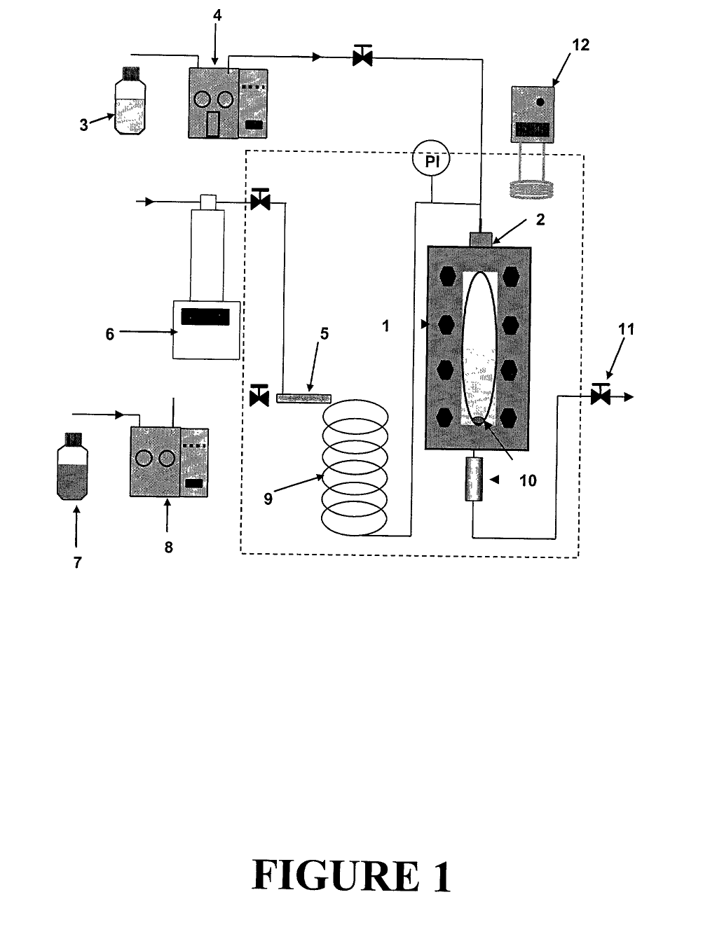

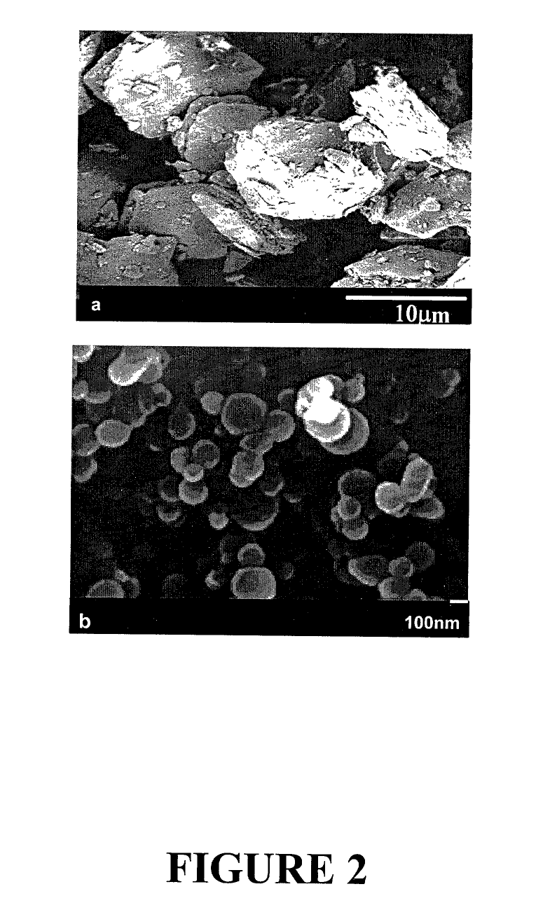

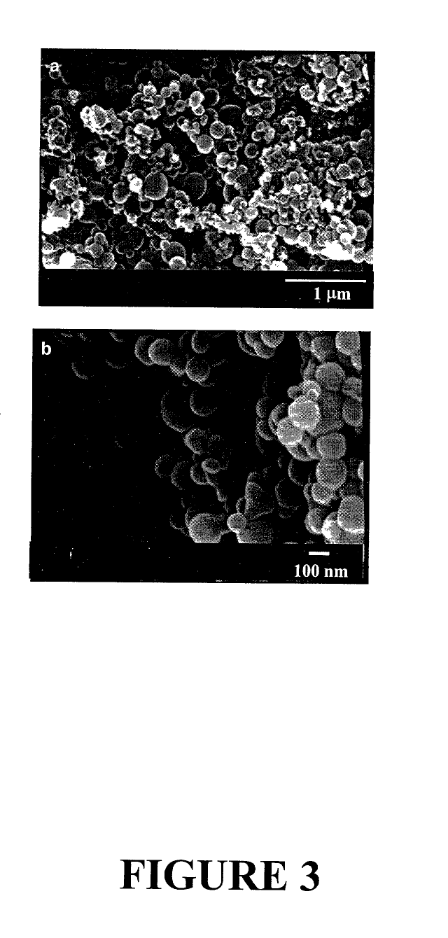

An ASES apparatus was used which is schematically shown in FIG. 1. The apparatus includes a precipitation vessel 1 which is fitted with a nozzle 2. Through this nozzle is sprayed a solution of the substance of interest and the dense gas and the modifier.

The solution of the substance of interest is pumped into the precipitation vessel from the reservoir 3 by means of pump 4. The dense gas is pumped into a static mixer 5 by means of pump 6. The modifier is simultaneously pumped from its reservoir 7 into the static mixer 5 by means of pump 8.

On leaving the static mixer, the dense gas/modifier mixture is passed through a cooling coil 9, and then into the precipitation vessel 1. The flow of the fluids continues through the precipitation vessel and through the filter 10.The flow rate is controlled by the metering valve 11. The apparatus is placed in a water bath, which is heated by the heater 12, to control and maintain the temperature of the precipitation vessel.

The desired mixture of ethanol with ethane is prepared in the static mixer 5. The vessel is first pressurised with carbon dioxide via a syringe pump (ISCO Model 500) 6 to attain a pressure of 20 to 180 bar to maintain the ethane/ethanol mixture as a single phase. The modified ethane is then delivered into the precipitation vessel 1 at the desired processing conditions and CO₂ is purged from the system. The operating temperature is controlled to within ±0.1° C. using a temperature controlled water bath heated by heater 12.

Once the desired temperature and pressure (namely 25° C. and 150 bar) are achieved in the vessel 1, and the vessel filled with 30 mol % ethanol in ethane mixture, the aqueous solution containing the protein (low endotoxin bovine insulin (lyophilized powder of 28.5 USP units/mg, lot No. 47H0573, purchased from Sigma) is pumped from reservoir 3 at a constant flow rate using a solvent delivery unit (Waters pump, Model 510) 4 and sprayed through a capillary nozzle 2 (50 um internal diameter) into the chamber. The pressure drop through the nozzle was adjusted to about 50 bar by a metering valve 21. This pressure drop can be adjusted to optimise the efficiency of the process. Modified ethane was fed continuously through to the chamber at a constant flow rate that was adjusted with the metering valve 11 placed at the exit.

After precipitation, water and ethanol residues in the protein were washed out at the operating pressure by modified ethane. The precipitation chamber was then gradually depressurised and the powder was collected for characterization.

The operating conditions, the flow-rate ratio of the aqueous feed and the anti-solvent, and the modifier mole fraction were optimized using ternary phase equilibrium data to have a homogenous mixture of ethane-ethanol-water in the precipitation vessel.

The mole fraction of ethanol in the anti-solvent was kept at 0.3 and a volumetric flow rate ratio of feed to anti-solvent of 0.4/12 was used in the process. This is primarily adjusted so as to maintain a single homogeneous phase in the system, particularly in the precipitation vessel. The high flow rate of the anti-solvent facilitated the dispersion and mixing of the aqueous spray mist across the chamber resulting in higher rates of water extraction from the droplets.

Micronised particles of proteins with uniform particle size suitable for aerosol drug delivery systems can now be produced from aqueous solution at room temperature in one step.

In the embodiment described, no toxic chemicals were used. We have found that the residual ethanol content in the final product to be less than 10 ppm.

The small particulate material of the present invention is particularly useful in the preparation of pharmaceutical preparations formulated to provide oral, controlled or sustained release, or for inhalation or transdermal administration and conventional modes.

### Example 2

### Modified ASES Apparatus

Procedure

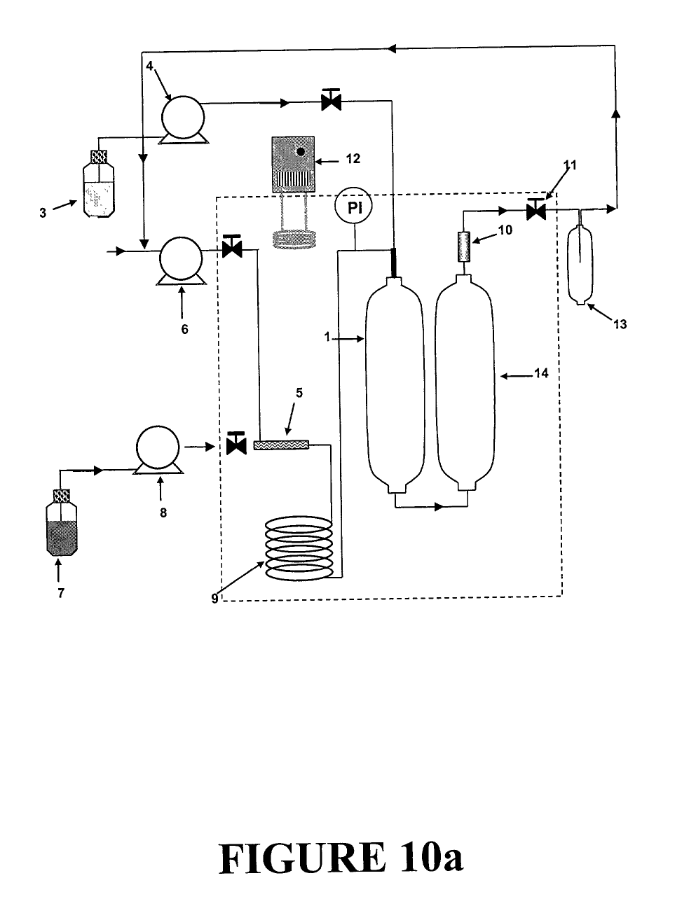

The schematic diagrams of the common and modified ASES apparatus are shown in FIGS. 9a, 9b, 10a, 10b and 11, respectively. The main part of the set-up is the high pressure vessel that in the previously described apparatus (FIG. 1) consists of only one vessel with a filter at the bottom to separate and collect the precipitate. In the modified design (FIGS. 10a and 10b), a second high-pressure vessel (a particle collection vessel) is added with no filter between the two vessels, but the filter is downstream of the particle collection vessel.

Referring to the modified ASES apparatus illustrated in FIGS. 10a and 10b, the apparatus includes a precipitation vessel 1 which is fitted with a nozzle 2. Through this nozzle is sprayed a solution of the substance of interest and the dense gas and the modifier.

The solution of the substance of interest is pumped into the precipitation vessel from the reservoir 3 by means of pump 4. The dense gas is pumped into a static mixer 5 by means of pump 6. The modifier is simultaneously pumped from its reservoir 7 into the static mixer 5 by means of pump 8.

On leaving the static mixer, the dense gas/modifier mixture is passed through a cooling coil 9, and then into the precipitation vessel 1. The flow of the fluids continues through the precipitation vessel and then into the collection vessel 14. The flow of fluids then passes through the filter 10. The flow rate is controlled by the metering valve 11. Once passing through the valve, the flow of fluids passes through a cold trap or separator 13, at low temperature, to separate the solvent and modifier from the dense gas. The dense gas can then be recycled through the system. The apparatus is placed in a water bath, which is heated by the heater 12, to control and maintain the temperature of the precipitation vessel.

FIG. 11 shows a modified design incorporating two particle collection vessels. The apparatus operates as described for FIGS. 10a and 10b, but once the first particle collection vessel has been filled to capacity, the flow of dense gas containing the formed particles can be diverted into a second particle collection vessel. The removal of the particles from the first particle collection vessel can be effected while the apparatus, particularly the precipitation vessel, is still in operation. Once the second particle collection vessel has been filled to capacity, the flow of dense gas containing the precipitated particles can be diverted into a third particle collection vessel, or back to the first particle collection vessel which by this time would have been cleaned out. In this way, the apparatus can operate in a continuous manner.

On the laboratory scale, the view cell (such as a Jerguson sight gauge, model 13-R-32) can be used as a precipitation vessel for visual observation of the precipitation stages. A coaxial nozzle is connected to the vessel for spraying the solutions and anti-solvent. The nozzle consists of a capillary tube (SGE, PEEK tube 200 mm length, 50 μm i.d., and 1.59 mm o.d.) inserted into a stainless steel tube (Alltech, 2.16 mm i.d. and 3.18 mm o.d.). The three pumps in the system are for the delivery of the protein solution (Waters Model 510), ethanol (Hewlett Packard, series 1050) and anti-solvent such as ethane and CO₂ (ISCO Syringe pump 500D). The anti-solvent was mixed with ethanol in line using a static mixer (KOFLO Corporation). The composition of the mixture was adjusted by the flow rate of each pump. The anti-solvent flow rate was controlled by a metering valve at the exit. The ethanol/water/anti-solvent mixture was maintained in a homogeneous phase at the operating pressure and temperature of the process. The carbon dioxide/ethanol mixture was passed through a preheating coil to attain the system temperature. The high pressure vessels were placed in a water bath consist of a thermostatic heater (Thermoline Unistat heater/circulator) to control the temperature.

The pressure of the system was monitored with Druck pressure transducers (Model PDCR 911) coupled to Druck pressure indicators. The anti-solvent and solvent were separated after the metering valve and the solvent was collected in a vessel. The filters were placed after the high pressure vessel to collect any remaining fine powder in the line.

Preparation of Particles

The preparation of the particles was carried out using the apparatus represented in FIG. 10b (discussed above). The desired mixture of ethanol with ethane is prepared in the static mixer 5. The vessel is first pressurised with carbon dioxide (which aids apparatus sterilisation) via a syringe pump (ISCO Model 500) 6 to attain a pressure of 20 to 180 bar to maintain the ethane/ethanol mixture as a single phase. The modified ethane is then delivered into the precipitation vessel 1 at the desired processing conditions and CO₂ is purged from the system. The operating temperature is controlled to within ±0.1° C. using a temperature controlled water bath heated by heater 12.

Micronisation by the ASES process was conducted by first placing the high pressure vessels in the water bath and adjusting the temperature of the system. After the system approached the temperature of the process, the pressure of the system was adjusted by adding anti-solvent to the vessels from the top. The anti-solvent flow rate was then adjusted by the metering valve at the exit. The required amount of ethanol was then added to the system by controlling the flow rate of each line. When using CO₂ as the anti-solvent, to achieve a CO₂-20 mol % ethanol mixture, a flow rate of 3.4 mL/min and 15 mL/min of ethanol and CO₂, respectively, were passed through the static mixer. When using ethane as the anti-solvent, an ethane-30 mol % ethanol mixture was prepared using flow rates of 15 mL/min and 2.4 mL/min for ethane and ethanol, respectively.

After the system approached steady state, 0.1 mL/min insulin solution (100 mg/mL) was sprayed through the inner nozzle. The solution dispersed due to the high flow rate of the anti-solvent. Extraction of the water was facilitated from the droplets by the ethanol and fine insulin particles were formed. After spraying the solution, ethanol and water residues were removed from the precipitate by passing the equivalent of 5 vessel volumes of ethane at operating pressure and temperature through the collection vessels. A small amount of ethane was also purged through the nozzle to remove any remaining solution. The metering valve at the exit is adjusted once precipitation commences such that the force exerted on the particles in the particle collection vessel by the dense gas flowing upwardly through the collection vessel is balanced by their weight (by gravity) so that the particles are in effect suspended within the collection vessel and not compacted. Other similar arrangements may be contemplated, such as collection vessels rotating about an axis to generate a force counter to that of the dense gas flowing through the collection vessel. The vessel is then depressurized and the product collected from both vessels, sealed in airtight containers and stored in the freezer (−18° C.) for characterization.

The effect of apparatus design on the characteristics of insulin particles precipitated from aqueous solution using ethane/30 mol % ethanol was examined at 25° C. and 155 bar. This is discussed further below.

### Example 3

It has been demonstrated that processing of insulin using ethane-ethanol provides for the retention of biological activity as indicated by the in vitro test for monomer content. Insulin processed with CO₂-ethanol experienced significant deactivation in this respect. In the single stage process (illustrated in Example 1 & FIG. 1) the particle characteristics of the CO₂-ethanol and ethane-ethanol processed material are similar as shown in Table 1.

TABLE 1
Data from Single Stage production unit

| Anti-Solvent | D_{(0.5, V)} | FPM (%) |
|---|---|---|
| Ethane-ethanol | 12.8 +/− 1.0 | 20.0 |
| CO₂-ethanol | 11.8 +/− 1.6 | 20.3 |

D_{(0.5,V)} Median particle size based on volume (i.e. below which 50% of particles occur.
FPM (%) Fine Particle Mass—mass fraction of particles below 5 μm according to Cascade Impactor tests.

In the dual stage process (illustrated in Example 2 and FIGS. 10a, 10b and 11) the fine particle mass for the CO₂-ethanol system is similar to that obtained for the single stage process. A dramatic increase in this parameter was obtained for material processed with ethane-ethanol.

Whilst some reduction in D_{(0.5,V)} occurs with the 2-stage CO₂-ethanol processing method, only 21.9% of this material can be dispersed to produce material less than 5 μm aerodynamic diameter. The material obtained using the 2-stage ethane-ethanol process has a dramatically reduced D_{(0.5,V)}, consistent with the 100% increase in the amount of material less than 5 μm aerodynamic diameter, as indicated in Table 2.

TABLE 2
Data from 2-Stage Process

| Anti-solvent | D_{(0.5, V)} | FPM (%) |
|---|---|---|
| Ethane-ethanol | 4.8 +/− 0.4 | 43.5 |
| CO₂-ethanol | 8.2 +/− 0.6 | 23.1 |

D_{(0.5,V)} Median particle size based on volume (i.e. below which 50% of particles occur.
FPM (%) Fine Particle Mass—mass fraction of particles below 5 μm according to Cascade Impactor tests.

### Example 4

### Collection of Insulin Particles from Example 2

By way of another comparison, insulin was precipitated in a single-stage apparatus as nano-sized particles, most of which ranged in size from 50-500 nm using the apparatus represented in FIG. 9b. Particle size distribution studies showed that the particles were agglomerated to form micron-sized particles. The fine particle mass of the micronised powder precipitated was only 20%. The particles were collected on the filter assembly at the bottom of the vessel. Due in part to the high pressure and high flow rate of the anti-solvent, the powder was compacted and difficult to disperse. The fine particles that were not aggregated washed from the system by passing through the filter, therefore the yield was low (40%).

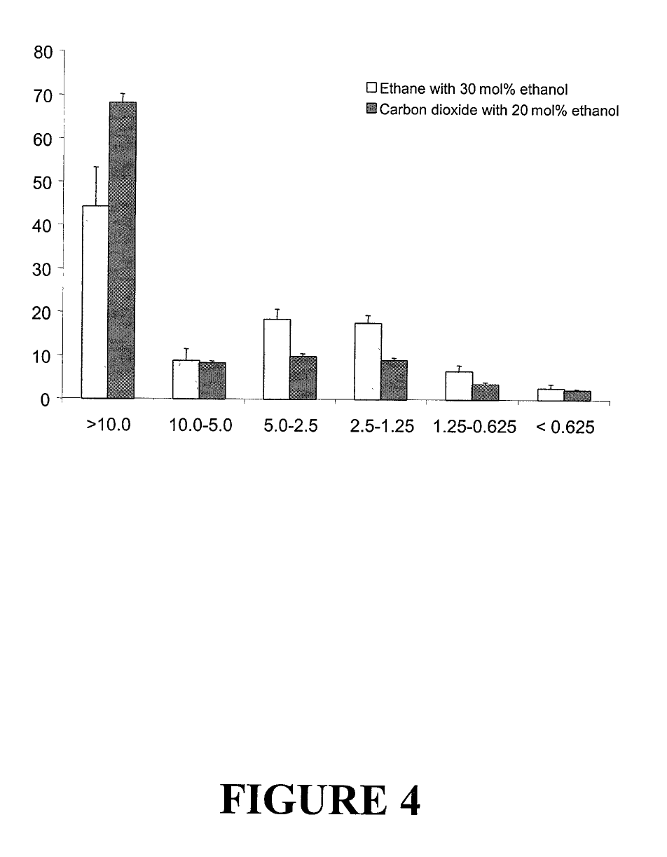

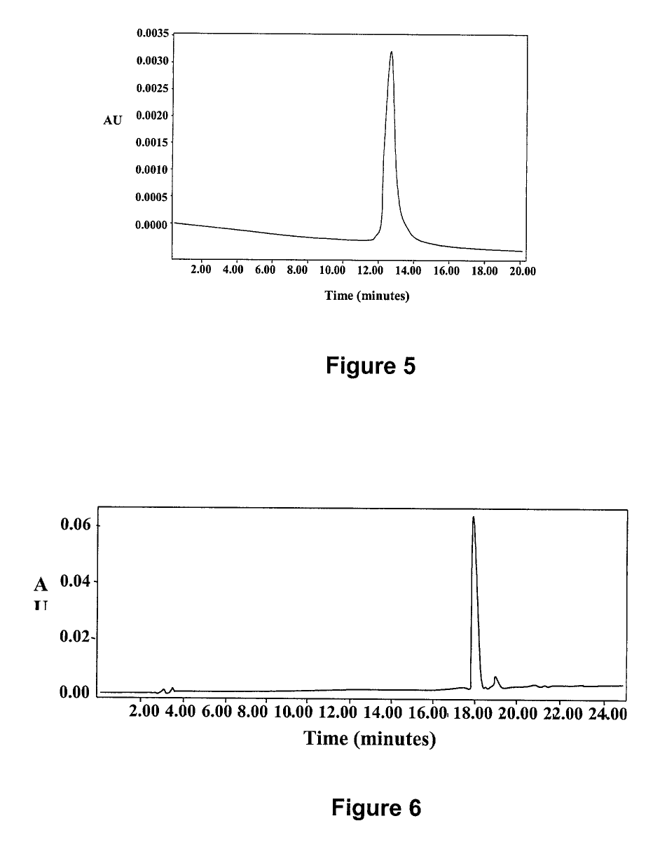

Insulin was precipitated as nano-sized particles ranged in size from 50-500 nm using the apparatus represented in FIG. 10b. The fine particle mass of insulin processed by the modified ASES apparatus was increased to 45%. The filter at the bottom of the first vessel was eliminated, producing a precipitate which was less compacted. The precipitate moves downward from the precipitation vessel and is carried upwardly part way through the collection vessel where gravity acts on it counter-currently to the anti-solvent (dense fluid) flow direction, suspending them in the vessel, which minimises the compaction of particles. Addition of the second (ie, particle collection) vessel enabled improved recovery of fine particle mass and increased the yield to 90%. FIG. 4 shows the aerodynamic particle size distribution of insulin powders in the single chamber and the dual chamber apparatus.

### Example 5

### Characterisation of Insulin Powder

Biochemical Integrity of Insulin Powder

In order to determine whether the biological activity of the insulin was retained after the particle formation process, the biochemical integrity of the insulin powder was assessed using size exclusion chromatography. A protein-Pak 125 column (Waters, USA) was used for insulin. The mobile phase consisted of 50 mM sodium phosphate buffer at pH 3 with 300 mM sodium chloride.

The powder samples were dissolved to prepare 1 mg/mL solution in deionised water by gentle shaking for 10 minutes to have complete dissolution. The supernatant was filtered through 0.45 mm membrane filter and then injected into the HPLC column.

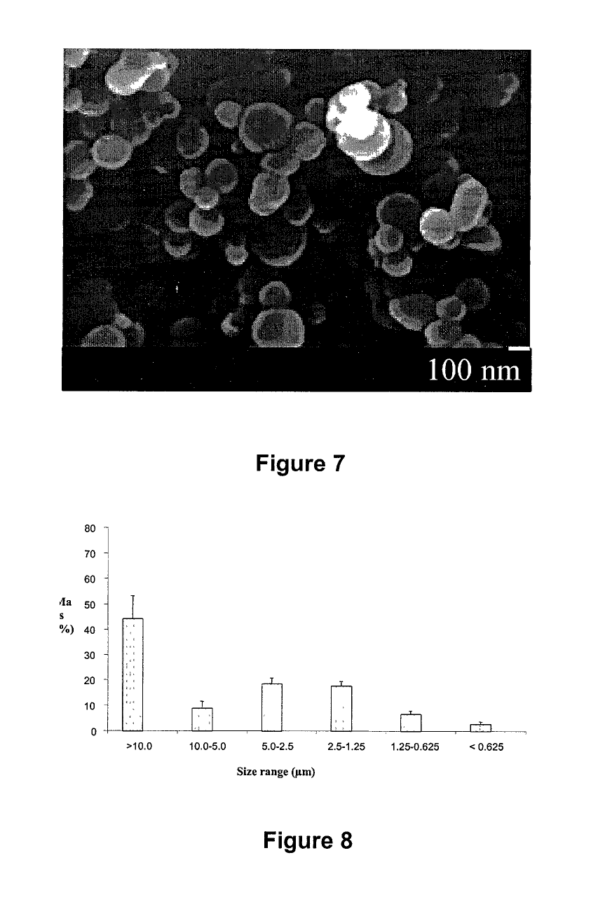

The percent of monomer and the soluble aggregates were determined by comparing the peak area of both monomer and the high molecular weight soluble species in the sample with the peak area of a standard concentration of 1 mg/mL The percent of insoluble aggregates was estimated from the difference in the total peak area between the sample and the protein standard solution. The software Millennium 3.5 was used in the quantification of the monomer content. The HPLC spectrum of insulin is shown in FIG. 6. The monomer content of the sample was 99.5% thus providing an in vitro measurement illustrating that retention of biological activity was almost complete.

Insulin Deamidation Testing

In order to determine the percentage of insulin that was deamidated by the process, a test of the insulin was performed using high performance liquid chromatography (HPLC). A reverse phase column (Symmetry® C₁₈, 5 μm packing, 4.6 mm×150 mm) was used to separate the deamidated from the non-deamidated insulin. The gradient method was used with eluents 0.1% trifluoroacetic acid/acetonitrile 26% to 33% and the flow rate of 1 mL/min. The absorbance was monitored at 280 nm.

The percentage of deamido insulin was determined by comparing the peak area of the degradation product to the area of standard insulin containing the same concentration of insulin (2 mg/mL).

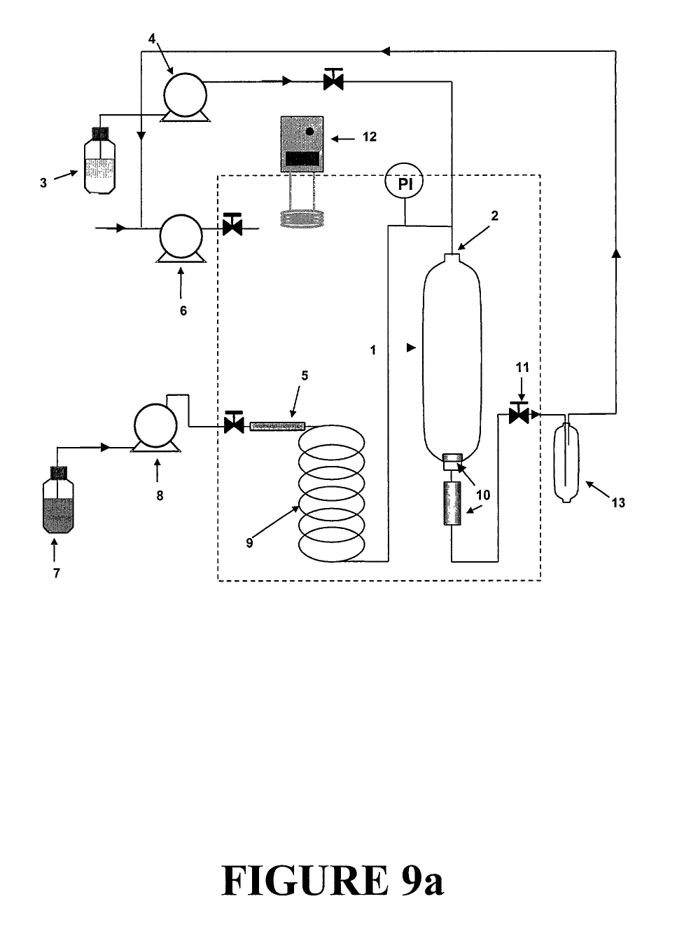

The HPLC chromatogram for the separation of insulin and deamido insulin is depicted in FIG. 7. The percentage of deamido insulin determined from the HPLC chromatogram was 3.0, thus providing additional in vitro evidence for the retention of biological activity and chemical integrity of the molecule after particlisation by precipitation.

Particle Size and Morphology

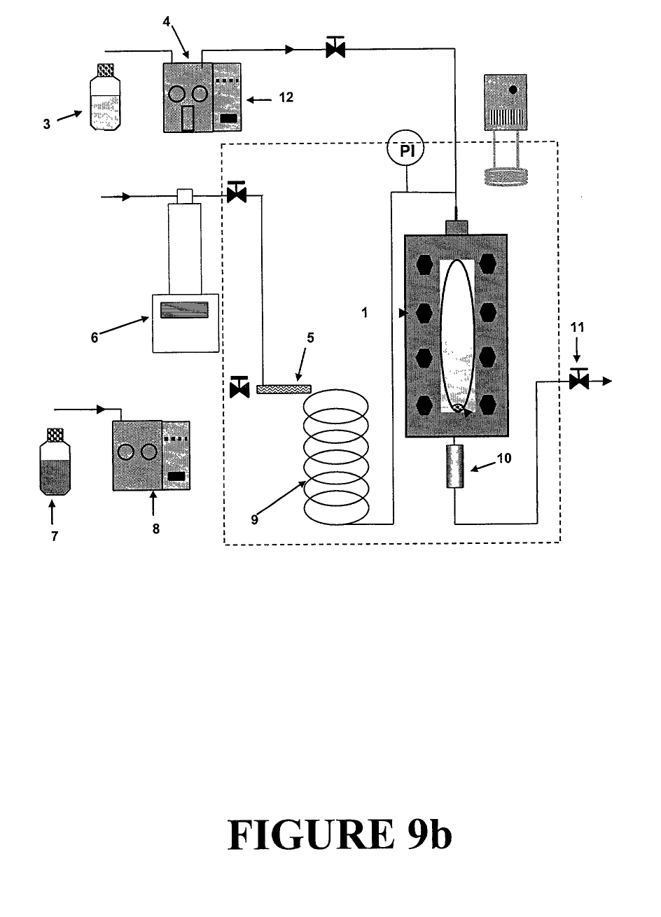

SEM images (FIG. 8) showed that insulin was precipitated as nano-sized particles ranging in size from 200 nm to 500 nm. These fine insulin particles may aggregate to form larger particles due to intermolecular interaction between the molecules. Analysing the particles by laser diffraction studies showed that the insulin powder agglomerated and possessed a median particle size of 5 μm (5000 nm).

Aerosol Characterisation by Impaction

In order to determine the aerosol performance of the protein particles, in vitro testing of the particles was assessed using a 5 stage Marple-Miller Impactor (Model 160, MSP Corporation, USA) with cut-off diameters of 10.0, 5.0, 2.5, 1.25, and 0.625 m. The Marple-Miller impactor consisted of a stainless steel inlet throat, five impaction stages and an integral filter stage. The stages were previously coated with propylene glycol/methanol (50:50) to prevent particle bounce, thus minimising the probability of carry over of coarse particles onto lower stages.

A mass of 10-20 mg of insulin was weighed into a gelatin capsule (size 3, Park Davis, Australia) and was immediately dispersed by a powder inhaler device, Dinkihaler (Aventis, Frankfurt, Germany) into the Marple Miller Impactor at 60 L/min using a vacuum source (ERWEKA GMBH, Germany).

The fraction of the protein recovered in each stage was calculated by measuring the absorbance of the protein solution against the absorbance of a standard 1% solution of insulin. The results of the assay method (measurement of relative UV absorption of a 1% solution) and the standard curves for insulin are indicated in Table 3.

TABLE 3
Protein assay methods and absorptivity value
determined from the standard curve.

| Protein | Assay method | Standard curve |
|---|---|---|
| Insulin | UV absorbance at 275 nm | A_{1%} = 9.0 |
| | | Conc. range = 0.01-0.1% |

Insulin precipitated as nano-sized particles, was also agglomerated to micron-sized particles. The median particle size (determined by laser diffraction) was about 5 μm and the fine particle fraction (% <5 μm), determined by the Marple Miller Impactor, was at least 42%, as shown in FIG. 8. The fine particle mass (FPM) achieved is significantly greater compared with the values reported in the literature. The maximum FPM for insulin obtained by other methods was 10%.

Preparation of Insulin Particles Using Ethanol/CO₂ as the Anti-Solvent

Finally, for comparison, an experiment was conducted as for example 2, using an aqueous solution containing low endotoxin bovine insulin and 20 mol % ethanol in CO₂ as the anti-solvent. The aerodynamic particle size distribution of the insulin powders produced is shown in FIG. 4. It can be seen that the fraction of particles with a size >10 μm produced using the ethane/ethanol mixture as the anti-solvent is around 45%, which is much lower than the fraction of particles with a size of >10 μm produced using carbon dioxide as the anti-solvent. Accordingly, the fraction of particles with a size of <10 μm is correspondingly higher in the case of the ethanol/ethane experiment than in the carbon dioxide experiment.

It will be understood that the invention disclosed and defined in this specification extends to all alternative combinations of two or more of the individual features mentioned or evident from the text or drawings. All of these different combinations constitute various alternative aspects of the invention.

The claims defining the invention are as follows:

1 A method for forming fine particles of a substance, the method including contacting a non-gaseous fluid containing the substance with a dense gas to expand the fluid, the dense gas including (a) an anti-solvent and (b) a modifying agent which modifies the polarity of the anti-solvent.

2 A method according to claim 1 wherein the anti-solvent does not significantly alter the pH of the non-gaseous fluid.

3 A method according to claim 2 in which the substance is pH-sensitive.

4 A method according to claim 2 in which the substance is biologically active.

5 A method according to claim 4 in which the modifying agent both modifies the polarity of the anti-solvent and acts as an extractant for the non-gaseous fluid.

6 A method according to claim 2 in which the anti-solvent is selected from the group consisting of a C₁₋₄ alkane gas, a C₂₋₄ alkene gas, a C₂₋₄ alkyne gas, refrigerant RF134a, or two or more thereof.

7 A method according to claim 6 in which the anti-solvent is ethane.

8 A method according to claim 6 in which the modifying agent is selected from the group consisting of C₁₋₆ alkanols, C₁₋₆ thiols and C₁₋₆ amines.

9 A method according to claim 8 in which the modifying agent is ethanol.

10 A method according to claim 9 in which the non-gaseous fluid is an aqueous solution and sufficient modifying agent is used to extract substantially all of the non-gaseous fluid to facilitate precipitation of the substance.

11 A method according to claim 1 in which the anti-solvent and modifying agent are maintained as a single phase.

12 A method according to claim 11 in which the non-gaseous fluid containing the substance and the dense gas are maintained as a single phase.

13 A method according to claim 11 in which the single phase Is maintained by either or both by adjustment of the temperature and pressure of the dense gas and by controlling the relative flow rates of each prior to expansion of the fluid.

14 A method according to claim 1 in which the dense gas is between 5° C. and 40° C. and at a pressure of between 5 to 150 bar.

15 A method according to claim 1 in which the substance is selected from the group of proteins, nucleic acids, liposomes, lipids (including phospholipids), water soluble polymers, controlled-delivery coatings, surfactants and phytosterols, whether natural or synthetic.

16 A method according to claim 1 in which about 50% of the particles formed are between 625 and 10,000 nanometers across.

17 A method according to claim 1 in which over 50% of the particles formed are less than 10,000 nanometers across.

18 A method according to claim 1 in which over 50% of the particles formed are smaller than 6,500 nanometers.

19 A method according to claim 1 in which the anti-solvent and modifying agent are combined before being contacted with the non-gaseous fluid.

20 A method according to claim 12 in which the concentration of the substance in the non-gaseous fluid is adjusted to maintain a single phase between the non-gaseous fluid/substance and the anti-solvent/modifying agent.

21 Fine particles of a substance formed by a method according to claim 1.

22 Fine particles according to claim 21 in which the particles comprise primarily biologically active insulin.

23 Fine particles according to claim 22 in which at least 50% of the particles are less than 10,000 nanometers across.

24 A pH-sensitive, biologically active substance in the form of fine particles produced by a method according to claim 1.

25 A method of treatment of a subject comprising administering to the subject an effective amount of fine particles according to claim 21.

26 A method according to claim 25 in which insulin-dependent diabetes is treated by administration of insulin particles.

27 Fine particles, each having a substantially similar shape and size according to claim 21.

28 A method according to claim 1 wherein the fine particles flow with the dense gas from a first vessel in which the particles are formed to a second collection vessel, from which the particles are collected.

29 A method according to claim 28, the second collection vessel having an inlet and an outlet disposed above the inlet, in which the fine particles and dense fluid pass through the inlet and the flow of dense fluid through the outlet is adjusted to maximize the proportion of fine particles collectable from the second collection vessel.
